(19) United States
(12) Patent Application Publication
(10) Pub. No.: US 2023/0405371 A1
(43) Pub. Date: Dec. 21, 2023

(54) DUAL LAYERED NANOPARTICLE COATED MASKS FOR EFFICIENT AND COST-EFFECTIVE FILTRATION OF AIR POLLUTANTS AND VIRUSES

(71) Applicant: Ishika Nag, Oviedo, FL (US)

(72) Inventor: Ishika Nag, Oviedo, FL (US)

(21) Appl. No.: 17/836,652

(22) Filed: Jun. 9, 2022

(51) Int. Cl. A62B 23/02; A01N 59/16; A01P 1/00; A01N 59/20; B01J 20/20; B01J 20/06; B01J 20/10; B01J 20/28; A61L 9/01

(52) U.S. Cl. CPC A62B 23/025; A01N 59/16; A01P 1/00; A01N 59/20; B01J 20/20; A61L 2209/14; B01J 20/103; B01J 20/28035; B01J 20/28028; B01J 20/28007; A61L 9/01; B01J 20/06

(57) ABSTRACT

A combination of nanoparticles applied onto the different layers of a mask filtration system, exemplarily including 1%-10% silicon dioxide on the outer layer of the mask and 1%-10% graphene; 1%-10% titanium dioxide; 1%-10% zinc oxide; 1%-10% copper oxide on the middle layer of the mask; and balance solvent is provided.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

Several waves of the different mutations of the SARS-CoV-2 virus have demonstrated that face masks are an important part of personal protective equipment (PPE), not only for the frontline health-care workers but for the general population as well. This has also resulted in severe and acute shortages in efficient and cost-effective masks. In the early days of the pandemic, healthcare facilities ran out of PPE and were forced to reuse single-use masks for days on end. Furthermore, the vulnerability to the most severe impacts of coronavirus outbreaks such as SARS in 2003 and SARS-CoV-2 in 2019 have been found to be correlated with long term exposure to air pollution. An increase of only 1 μg/m3 in PM_{2.5}, particulate matter less than 2.5 μm in size, is associated with an 11% increase in the COVID-19 death rate.

Ninety-nine percent of the world's population live in places exceeding the World Health Organization's air quality guidelines of 10 μg/m³ and 7 million people die every year because of air pollution. The most polluted areas around the world tend to be in developing countries and communities with limited resources such as in South-East Asia, Africa, and China, due to their increased density of urban population, significant use of fossil fuels and relatively inadequate control measures. Hence, access to effective and affordable filtration devices have remained a challenge.

The safety of nanoparticle usage is of utmost importance and continues to be a subject of research worldwide. A careful selection of nanoparticles with attention to their permissible exposure limits, as per OSHA standards is critical, especially for application on face masks.

### FIELD OF THE INVENTION

The invention is directed generally to air pollution prevention and viral particle abatement in filtration systems and more particularly to a system of specific nanoparticles applied in a specific layering technique to a commonly available filter or face mask and enhancing its particle and virus filtration capabilities.

### SUMMARY OF THE INVENTION

An objective of this invention is to develop an engineered face mask with selective and optimized nanoparticle layering for filtration of air pollutants like PM_{2.5} and viral pathogens like SARS-CoV-2.

Furthermore, the goal of this invention is to improve the Particle Filtration Efficiency (PFE) and the Virus Filtration Efficiency (VFE) of a regular mask, while demonstrating that the Nanoparticle Retention Efficacy (NRE) are within acceptable Permissible Exposure Limits (PEL), as defined by the Occupational Safety and Health Administration (OSHA).

Another objective of this invention is to develop a simple and versatile application technique of the nanoparticles such that it can be applied in different parts of the world, thus providing an affordable and comparable alternative to expensive PPE.

Another objective of this invention is to provide a combination of nanoparticles applied onto a filtration system, exemplarily including 0%-10% graphene; 0%-10% titanium dioxide; 0%-10% zinc oxide; 0%-10% copper oxide; 0%-10% silicon dioxide; and balance solvent.

### BRIEF DESCRIPTION OF THE DRAWINGS

FIG. 1 is a schematic of an embodiment of the multi-layer filtration system in a typical surgical mask.

FIG. 2 is a schematic of an embodiment of the selective nanoparticle mixture that is impregnated onto the outer and inner layers of the mask.

### DETAILED DESCRIPTION

Typical surgical face masks are made of an outer non-woven layer for moisture absorption, a middle melt-blown electret layer for adsorption and filtration and an inner soft non-woven layer for vapor/mist absorption. Particle entrapment in the middle layer is typically dominated by four mechanisms-inertial impaction, interception, diffusion, and electrostatic attraction. Diffusion and interception mechanisms are typically effective for particle sizes between 1 and 3 μm like PM_{2.5}. The outer layer of the mask advantageously captures human respiratory droplets—viral particles encased in moisture (typically in the range of 1-10 μm) by these mechanisms. Electrostatic filtration is facilitated by coulombic forces for oppositely charged particles and by electrostatic induction for charge-neutral particles.

Nanoparticles possess at least one dimension of 1 to 100 nanometers (nm; 1 nm=10−9 meter). Particles have diameter less than 100 nm exhibit new size-dependent properties compared with the bulk material. Nanoparticles exist in the natural world and are also created from human activities. For example, there are several types of engineered nanomaterials such as carbon nanotubes, nanocomposites, quantum dots, fullerenes, quantum wires, and nanofibers Because of the submicroscopic size of nanomaterials, nanomaterials have unique material characteristics, and manufactured nanoparticles may find practical applications in a variety of areas, including medicine, engineering, catalysis, and environmental remediation.

Nanoparticles have a high surface to volume ratio, which enhances the entrapment of particulate matter by adsorption. Some of the nanoparticles used for this experiment like graphene, titanium dioxide (TiO₂), and zinc oxide (ZnO), are known to have filtration properties due to their high adsorption capabilities. TiO₂ nanoparticles, with its photocatalytic properties, absorb the ultraviolet component of sunlight and act as a catalyst to form reactive hydroxyl (·OH) radicals and the superoxide anion (O₂·—) from atmospheric moisture and oxygen. These radicals react with the PM_{2.5} particles due to their strong oxidizing capabilities converting them into CO₂ and H₂O.

Metal based nanoparticles, like Copper Oxide and Zinc Oxide, have unique physico-chemical properties due to their small size and high specific surface area, which enable them to interact with viruses. Respiratory diseases such as COPD, bronchitis, and asthma lead to the overexpression of ACE-2 enzyme in human respiratory cells for viral attachment. The SARS-CoV-2 virus primarily attacks the respiratory tract. Its spike protein attaches to the over expressed ACE-2 receptors in the epithelial cells of the tract, thus increasing the severity of the respiratory disorders leading to fatalities. Metal-based nanoparticles generate Reactive Oxygen Species (ROS) which oxidize viral proteins and nucleic acids, such as the spike protein in SARS-CoV-2 virus. Other similar viruses include MERS, SARS, Influenza, and the like.

While many nanoparticles are known to have many beneficial properties, there is ongoing research on the harmful effects of certain nanoparticles. The selected nanoparticles for this application are known for their clinical safety and non-toxicity and are extensively used in cosmetic and biomedical applications e.g., pill coatings, sunscreens. The risk of nanoparticles being dislodged from the mask and inhaled were evaluated and found to be within 3% of the permissible exposure limit, as per OSHA standards. The dual layer coating improves this further to less than 1% of the PEL, even for regular and strenuous breathing cycles.

All the nanoparticles used, were in the 40-50 nm size range, but the size could be larger (e.g., 50-100 nm) or smaller (e.g., 1-40 nm). Ethanol was chosen as a solvent since it penetrates the fibrous filtration media in its liquid state, impregnates the nanoparticles in the pores of the filters and then evaporates at room temperature, but other solvents could be used including but not limited to water, methanol, acetone, chloroform, and dichlorobenzene. Different application mechanisms were also tried—pipette, spray bottle, pressurized sprayer system, and airbrush sprayer—to determine the most uniform spatial distribution, the best durability and the most nanoparticle retention efficacy (NRE), although other application mechanisms could be used. The airbrush sprayer system which aerosolizes the ethanol-nanoparticle mixture, was found to penetrate the outer layers of the masks and gets the nanoparticles impregnated in the melt-blown middle layer of the masks.

The dual-layer coated masks used in this study are deposited with SiO₂ nanoparticles on the outer layer to enhance adsorption of moisture encased virions with its desiccant properties and its large specific area. The middle layer of these masks is deposited with graphene, TiO₂, CuO and ZnO nanoparticles. Graphene, with its hexagonal lattice structure, promotes adsorption and acts as a platform for other nanoparticles. TiO₂ generates the superoxide anion and OH radicals in presence of light which oxidizes the particulate matter into CO₂ and H₂O. CuO increases the antiviral capability of TiO₂ by enhancing its photoactivity by lowering the bandgap of Cu—TiO₂, making the electron jump between bands quicker, especially in the visible light spectrum, since TiO₂ works better in the UV light spectrum. ZnO and CuO are known for their ability to generate reactive oxygen species (ROS) which oxidize viral proteins and nucleic acids, such as the spike protein in SARS-CoV-2 virus.

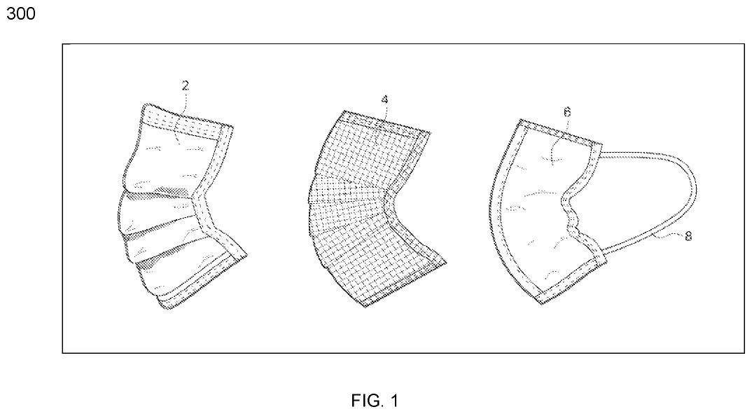

As seen in FIG. 1, a typical surgical mask comprises of an outer layer 2, a middle layer 4 and an inner layer 6 and a pair of ear loops 8 to hold the mask in place.

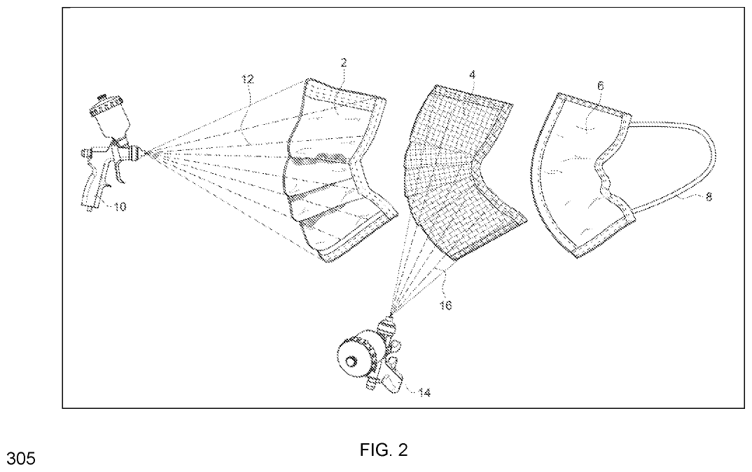

A 1-50%, preferably 1-20%, and most preferably 6-8%, mixture of nanoparticles (weight by weight) was dissolved in the ethanol medium. This solution was then aerosolized using the pressurized sprayer system 10 and 14, as seen in FIG. 2. The aerosolized spray 12 was directed towards the mask while maintaining a spray-distance of about 6-8 inches. The mask was then air-dried for at least 8 hours and then tested for efficacy.

A layered deposition technique was applied by cutting the mask open from its periphery and applying selective nanoparticles in the outer and middle layers of the masks, as seen in FIG. 2. SiO₂ nanoparticles were deposited on the outer non-woven layer for its desiccant and adsorption properties 12, while the other nanoparticles (ZnO, TiO₂, Graphene, CuO) were deposited onto the melt-blown middle layer 16, to improve filtration efficiency and retention efficacy.

### Example I

### Selective Coating of Nanoparticles in Different Layers of the Mask

Several combinations of nanoparticles are possible with varying characteristics and were selected from. SiO₂ nanoparticles were deposited on the outer non-woven layer for its desiccant and adsorption properties, while the other nanoparticles (ZnO, TiO₂, Graphene, CuO) were deposited onto the melt-blown middle layer, to improve filtration efficiency and retention efficacy.

### Example II

### Synthesis of Nanoparticle Coatings by Varying Mixture Combinations and Concentrations

Several combinations of nanoparticles and their concentrations are possible within the ranges and were experimented with. A full factorial Design of Experiments (DOE) statistical analysis model was used to collect and analyze the data to randomize the run order of the experiment, minimize bias and aid with the Analysis of Variance (ANOVA) study. The 1-50%, preferably 4-10%, and most preferably 6-8%, mixture of nanoparticles (weight by weight) researched and selected as being particularly effective for purposes of this invention include without limitation.

### Example III

### Analysis of the Effectiveness of the Nanoparticle Coated Masks for Particle Filtration Efficiency

A wind tunnel was used to test for the particulate filtration efficiency (PFE) of the masks at ambient temperature (25° C.+/−5° C.) conditions. A plexiglass enclosure (122×61×30.5 cm) used as the body of the tunnel, with an aluminum exhaust tube (Imperial, 10.2 cm×244 cm) connecting the PM_{2.5} source (incense sticks, wood chips, paraffin) to the inlet section of the tunnel. A fan (Holmes HAOF 85, 23×23 cm) was placed inside the tunnel, to create the draft. Two soft silicone mannequin heads (Yephets, 23×15×10 cm), one with a nanoparticle coated mask and another control mannequin without a mask were tested side by side with vacuum pumps (HSH-Flow, 6 W, 8 LPM, 120 KPa) simulating human breathing. Laser particle detectors (Temtop, LKC-1000S) were connected by plastic tubing (Ø 0.64 cm) and funnels (Ø 7 cm) to the mannequins and to the vacuum pump, to measure PM_{2.5}.

PFE was evaluated (1) by measuring the flow rate (mg/m³) of PM_{2.5}, of the masked mannequin (φ2) and the control mannequin without mask (φ1); while both mannequins were placed side-by-side and exposed to the same environment.

$$PFE ( \% ) = \frac{{φ}_{1 -} {φ}_{2}}{{φ}_{1}} \times 100 \qquad ( 1 )$$

$${φ}_{1} = {PM}_{2.5} without mask \\ {φ}_{2} = {PM}_{2.5} with mask$$

### Example IV

### Analysis of the Effectiveness of the Nanoparticle Coated Masks for Virus Filtration Efficiency

Virus filtration efficiency (VFE) was tested with nebulized NaCl particles as a surrogate for virus charged respiratory droplets, per the test protocol recommended by the Center of Disease Control (CDC). A mannequin connected to the nebulizer (Mayluck, 0.25 ml/min atomization rate, 0.5-10 μm particle size) ‘exhaled’ the NaCl particles (0.9% saline solution made from distilled water and table salt) which were ‘inhaled’ by the mannequin wearing the nanoparticle coated mask. The mannequins were kept 25 cm apart and the exposure time was 20 mins per mask, in order to deposit sufficient NaCl particles in the collection chamber to be quantified by the gravimetric measurement procedure. The set-up was housed in a plexiglass enclosure (122×30.5×30.5 cm) to protect against wind draft and maintain consistency. The VFE was calculated (2) by comparing the weight of NaCl deposited on the fine filter paper after being inhaled through the mask (γ2), to the control case without mask (γ1).

$$VFE ( \% ) = \frac{{γ}_{1 -} {γ}_{2}}{{γ}_{1}} \times 100 \qquad ( 2 )$$

$${γ}_{1} = NaCl ( virus surrogate ) inhaled without mask \\ {γ}_{2} = NaCl ( virus surrogate ) inhaled with mask$$

### Example V

### Analysis of the Nanoparticle Coated Masks for Nanoparticle Retention Efficacy

NRE was used to evaluate whether the dislodged nanoparticles from the mask, if inhaled, were still within the Permissible Exposure Limit (PEL) as specified by OSHA. As seen in (3), the PEL Utilization was calculated by comparing the weight of nanoparticles dislodged from the mask during an 8-hour operational period (ωᵢ) to the PEL limit for that nanoparticle (ω_{iPEL}) and taking a weighted average of the nanoparticles embedded in that mask. The total weight of all the nanoparticles collected was measured using the gravimetric method with a micro-balance (0.1 mg accuracy), as recommended in the CDC test procedure and the individual weights of the nanoparticles were calculated by the ratio of their molecular weights.

$$PEL Utilization ( \% ) = \frac{1}{n} \times {\sum }_{i = 1}^{n} \frac{{ω}_{i}}{{ω}_{{i}_{PEL}}} + {\sum }_{j = 1}^{n} \frac{{\dot{v}}_{j} \times {t}_{j} \times 60}{1000} \times 100 \qquad ( 3 )$$

$$n = number of NPs in mask \\ i = NP type \\ {ω}_{i} = weight of {NP}_{i} inhaled from mask [ mg ] \\ {ω}_{{i}_{PEL}} = Permissible Exposure Limit for {NP}_{i} [ mg / {m}^{3} ] \\ {v}_{j} = Breathing flow rate for time j [ lpm ] \\ {t}_{i} = time at this breathing rate [ hours ] \\ PEL = Permissible Exposure Limit per OSHA standard$$

### Example VI

### Analysis of the Photocatalytic Activation of Nanoparticles

The photo-catalytic activation of nanoparticles to different kinds of light was tested by simulating daylight with an electrical lamp (Hyperikon, 15 W, 5000K) and a UV-A lamp (385-400 nm) was used to activate the photocatalytic properties of the nanoparticles. A main effects analysis method from the statistical ANOVA analysis method, was used to show that the statistically significant dependence of PFE with light exposure. PFE increased by 7% with exposure to daylight and by 13% in presence of UV light.

### Example VII

### Analysis of the Spatial Uniformity of Nanoparticle Deposition

The deposition method of nanoparticles onto the filtration media were also varied and tested for uniform spatial distribution. Different spray mechanisms were tested using pipettes, spray bottles, pressurized sprayers, and airbrush sprayers. The differently sprayed filtration media were tested using the particulate filtration efficiency method to analyze the efficiencies of the deposition methods. The airbrush spray application resulted in the most uniform spatial distribution of the nanoparticles and was chosen as the preferred application method for its simplicity and effectiveness.

### Example VIII

### Analysis of the Surface Morphology of Nanoparticle Coatings

The surface morphology of the masks was characterized using the scanning electron microscopy (SEM) imaging technique. A Zeiss Ultra-55 SEM with a Schottky field emission source and resolution of 1 nm at 15 KV, 1.7 nm at 1 KV was used. The images of three typical surgical masks; one without any coatings, one with nanoparticle coatings before and one after exposure to PM_{2.5}, were compared at 1000× magnification.

The SEM images illustrated the differences in fiber size and density of the inner and outer layer of the surgical masks. The middle layer has much thinner fibers (300 μm diameter) randomly oriented to enable the impaction and diffusion mechanisms of particle entrapment. The images from the coated masks confirmed the nanoparticle adhesion to the fibers of the masks in both the outer and middle layers. The images from the masks exposed to PM_{2.5} confirmed the entrapment of particulate matter onto the nanoparticle surfaces of the coated masks, especially in the enhanced view with 5000× magnification. The SEM images confirmed the adhesion of the embedded nanoparticles and the subsequent entrapment of the PM_{2.5} particles due to the high surface to volume ratio and photocatalytic activation properties.

### Example LY

### Analysis of the Measurement System Repeatability and Reproducibility

A statistical repeatability and reproducibility study (Gage R&R) was used, using Minitab analytical software, to determine the measurement uncertainty of the experiments. For the PFE set-up, four different mask types were tested with ten repetitions each, on three different days. It was seen that 97% of the contribution was from ‘part-to-part variation’ which is contributed by the natural process variation of the different masks and their coatings while only 3% of the variation is contributed by the repeatability (one mask tested multiple times) and reproducibility (one mask tested over different days). The total Gage R&R being 3% is deemed to be an acceptable measurement uncertainty.

### Example X

### Analysis of Pressure Drop and Breathability

The pressure drop measurement for masks is a critical functional parameter affecting the breathability and comfort in wearing the mask. The pressure drop can be calculated by using the Bernoulli equation (4):

$${P}_{1} + \frac{1}{2} ρ {v}_{1}^{2} + ρ g {1}_{} = {P}_{2} + \frac{1}{2} ρ {v}_{2}^{2} + ρ g {2}_{} \qquad ( 4 )$$

where P is the pressure, ρ is the density of the fluid, ν is the velocity, g is the gravitational acceleration, h is the height, subscript₁ denotes upstream conditions and subscript₂ is for downstream conditions. Under steady, incompressible, and frictionless flow along a streamline assumption with the same horizontal height; (4) can be simplified to the pressure drop equation (5):

$$Δ P = \frac{1}{2} ρ ( {v}_{1}^{2} - {v}_{2}^{2} ) \qquad ( 5 )$$

The pressure drop was measured using a manometer, with the tubes placed upstream and downstream of the masked mannequin. The results indicate that all the measured masks were within the acceptable guidelines of the pressure drop requirements as specified by the CDC.

Furthermore, the deposition of nanoparticles did not have a significant or measurable impact on pressure drop of the masks. This minimal impact is expected, since the size of the nanoparticles (15-45 nm) are significantly smaller than the pore size of the masks (typically 10-90 μm) and hence do not cause a significant blockage effect that could have an impact on pressure drop.

### Example XI

### Analysis of the Viral Load on the Masks

Studies on respiratory droplet sizes have indicated particle sizes between 0.8 to 5 μm, where breathing produced droplet sizes of 0.8 μm at an average concentration of 0.1 cm⁻³ while vocalization (speech) produced droplet sizes between 3.5-5 μm at an average concentration of 1.1 cm⁻³. The nebulizer used for this study produced aerosolized droplet sizes with a median mass aerodynamic diameter (MMAD) of 5.3 μm, which correlates well with typical sizes of aerosolized respiratory droplets, carrying different virions. The typical particle concentration, for breathing or vocalization, is characterized between 0.1-1.1 cm⁻³ at a typical exposure of <1 second. The particle concentration exposure (viral load) in this experiment (ρᵢ), is calculated using the volume flow rate of the nebulizer, the MMAD of the nebulized particles, the time of exposure and the volume of the enclosure (6).

$${ρ}_{i} = \frac{{\int }_{0}^{t} \dot{ϑ} dt}{4 / 3 π {r}_{i}^{3}} \times \frac{1}{V} \qquad ( 6 )$$

$${ρ}_{i} = NaCl ( i ) particle concentration = 5.64 \times {10}^{5} [ {cm}^{- 3} ] \\ \dot{ϑ} = volume flow rate of nebulized NaCl solution = 0.25 [ \frac{{cm}^{3}}{min} ] \\ t = time of exposure = 20 [ min ] \\ η = Median mass aerodynamic radius of NaCl particles = 2.65 [ μ m ] \\ V = Volume of VFE experimental enclosure = 1.135 \times {10}^{5} [ {cm}^{3} ]$$

The calculated particle concentration at >5×10⁵ cm⁻³ can be translated into the total exposure, which is more than 1200 times of the typical exposure. However, the results may need to be adjusted for respiratory particle size, longevity, climatic conditions etc.

As will be understood by those skilled in the art, the selection and concentration of nanoparticles can be further adjusted to optimize durability, uniformity, and retention efficacy. Similarly, the selection and concentration of nanoparticles can be based on filtration efficacy, biocidal capabilities, and clinical safety. Additionally, the deposition method can be adjusted for spatial uniformity, penetration through the outer layers of the filtration layers and retention of the penetrated particles onto the fibrous layers of the filtration systems for continuous and effective operation. Also, the solvent selection can be adjusted, added to, changed, or replaced for desired benefits consistent with the invention.

Although exemplary embodiments of the present combination of nanoparticles applied onto a filtration system has been described in detail, those skilled in the art will understand that various changes, substitutions, variations, and improvements disclosed herein may be made without departing from the spirit and scope of the disclosure in its broadest form.

None of the description in the present application should be read as implying that any particular nanoparticle, element, step, act, or function is an essential element, which must be included in the claim scope: the scope of patented subject matter is defined only by the allowed claims.

I claim:

1. A mask filtration system, comprising:
an outer non-woven filtration layer-sized and configured to block human respiratory droplets and particles;
a middle melt-blown filtration layer sized and configured for electrostatic filtration;
an inner non-woven layer filtration sized and configured to absorb breathed vapor; and
a plurality of nanoparticles applied onto the outer, middle or inner filtration layers.

2. The mask filtration system of claim 1, wherein the plurality of nanoparticles comprise:
1%-10% graphene;
1%-10% titanium dioxide;
1%-10% zinc oxide;
1%-10% copper oxide; and
1%-10% silicon dioxide.

3. The mask filtration system of claim 1, wherein the plurality of nanoparticles comprise:
1%-10% silicon dioxide in the outer filtration layer;
1%-10% graphene, 1%-10% titanium dioxide, 1%-10% zinc oxide, or 1%-10% copper oxide in the middle filtration layer; and
no nanoparticles in the inner filtration layer.

4. The mask filtration system of claim 1, wherein the plurality of nanoparticles comprise:
1%-10% silicon dioxide or 1%-10% graphene in the outer filtration layer;
1%-10% titanium dioxide, 1%-10% zinc oxide, or 1%-10% copper oxide in the middle filtration layer; and
no nanoparticles in the inner layer.

5. The mask filtration system of claim 1, wherein the plurality of nanoparticles comprise:
1%-10% silicon dioxide, 1%-10% graphene, or 1%-10% titanium dioxide in the outer filtration layer;
1%-10% zinc oxide or 1%-10% copper oxide in the middle filtration layer; and
no nanoparticles in the inner layer.

6. The mask filtration system of claim 1, wherein the plurality of nanoparticles comprise:
1%-10% silicon dioxide or 1%-10% graphene in the outer filtration layer;
1%-10% zinc oxide or 1%-10% copper oxide in the middle filtration layer; and
1%-10% titanium dioxide in the inner filtration layer.

7. The mask filtration system of claim 1, comprising:
1%-10% silicon dioxide and 1%-10% graphene, in the outer filtration layer;
1%-10% copper oxide in the middle filtration layer;
1%-10% titanium dioxide and 1%-10% zinc oxide in the inner filtration layer.

8. The mask filtration system of claim 1, comprising:
1%-10% zinc oxide and 1%-10% copper oxide in the outer filtration layer;
1%-10% silicon dioxide and 1%-10% graphene, in the middle filtration layer;
1%-10% titanium dioxide in the inner filtration layer.

9. The mask filtration system of claim 1, wherein:
the mask filtration system filters airborne pollutants comprising PM_{2.5} and aerosolized virus particles selected from the group consisting of: SARS-CoV2, MERS, SARS, Influenza, and the like.

10. The mask filtration system of claim 9, wherein the mask is sized and configured to be worn by a human.
